(19) United States
(12) Patent Application Publication
(10) Pub. No.: US 2002/0095106 A1
(43) Pub. Date: Jul. 18, 2002

(54) Carpal tunnel support

(76) Inventors: Philip J. Caliguire, (US); Ella M. Caliguire, Cape Coral, FL (US)

Correspondence Address: FRANK A. LUKASIK, SUITE 142, 1250 W. MARION AVE., PUNTA GORDA, FL, 33950, US

(21) Appl. No.: 09/973,435

(22) Filed: Oct. 9, 2001

Related U.S. Application Data
(60) Provisional application No. 60/238,541, filed on Oct. 10, 2000.

(51) Int. Cl. A61F 13/00

(52) U.S. Cl. 602/41

(57) ABSTRACT

A carpal tunnel support for use by a wearer in the operation of components located above a work surface consisting of a 12″ flexible sleeve preferably made from nylon, and having a strip of hook and loop material fastened along its length and an elongated support panel having a strip of hook and loop material fastened along its length, the support panel is attached to the sleeve with the hook and loop material.

### RELATED APPLICATION

Claims priority of Provisional Patent Application Serial No. 60/238,541 filed Oct. 10, 2000

### BACKGROUND OF THE INVENTION

1. Field of the Invention

The field of the instant invention is wrist support devices generally and carpal tunnel syndrome preventives specifically.

Carpal Tunnel Syndrome (CTS) is a steadily increasing problem among workers who perform repetitive tasks generally, and among typists and other keyboard workers in particular.

The pain resulting from injury to the median nerve and the nine tendons that pass through the bony passageway through the wrist can be excruciating, very difficult to treat and almost impossible to fully cure.

Loss of productivity due to CTS among keyboard workers and even occasional keyboard users is estimated to be in the billions of dollars annually, and in some cases is so severe as to destroy a sufferer's ability to earn a livelihood at all.

A previous patent specification filed by one of the applicant inventors as U.S. Ser. No. 08/509491 described a resting arm splint that was proven efficacious in treatment of CTS by maintaining the wrist in a resting neutral position for extended periods of time at night, and contemplated that the wearer would regenerate hand and wrist functions through a resting period and then be able to remove the resting splint for a working period. In that invention, while effective for its design purpose, the bulk and extent of the semi-rigid battened splint from fingers to mid-forearm, prevented sufficient movement of the hand and fingers to be operable in a working environment, and specifically would interfere with keyboard operation. The further development of the within invention addresses the need for a CTS treatment modality that can relieve pain by restraint of the wrist joint to a neutral position, yet be compact and light enough to enable working functionality at a keyboard. Further, appliance for this purpose should be comfortable enough to be worn for long periods in a working environment and be easily donned or doffed.

Finally, if such a therapeutic appliance could be achieved, it would also operate as a preventive to further repetitive strain injury, and in some eases serve as a preventive if worn by a pre-injury user. While injured users may more readily be attracted to use of the within described carpal tunnel splint, the invention has been specifically designed to be attractive and comfortable in use so as to appeal to the pre-injury keyboardist, as it has been discovered that supporting and urging the wearer's hand and wrist to a neutral position is more comfortable and productive.

In this regard, the carpal tunnel splint incorporates a stiffening foam rubber batten that is necessary for therapeutic application to an injured user but optional to a pre-injury user, as that user finds the most comfortable mode of wear and usage in the operating environment. Comfort is an important consideration, since in the fast pace and urgency of the working environment, workers will shed uncomfortable, bulky or restrictive devices, no matter how therapeutic they may be.

While the majority of CTS complaints may result from keyboard work, other occupations also endure CTS or repetitive strain injuries and can benefit from the within invention. Contemplating other occupations, the device has been further designed to be adaptable to other working environments. The hook and loop (VELCRO) attachments of the present invention allow the user to adjust fit and placement of the support panel.

2. Description of the Related Art

Orthopedic restraint devices of many types and styles fill he annals of the prior art and commerce, some effective for some purposes and some not. Mere restrained movement is primary treatment for any joint injury or pain, and the present invention seeks to extend this tradition of joint restraint. However, particularized restraint to a specific position that best serves treatment of a particular joint on an individual sufferer must address precisely positioned restraint on a variety of sizes and shapes of human appendage, and this problem is compounded by the need to accommodate some movement in a working environment. Thus the device can strike a compromise between maximum restraint and user adjustable comfort, while still retaining a significant stiffness that will urge, not force, the user'wrist position to the neutral.

Many commercial products are now provided that claim CTS relief by merely wearing a close-fitting glove product that will put pressure on the joint and provide a modest limitation on movement, and these, while providing minor comfort, are generally ineffective to either relieve painful nerve contact within the carpal tunnel or prevent further repetitive stress injuries.

Others limit movement more severely by straps, buckles and rigid brackets and stays, which are often uncomfortable and also limit working effectiveness unacceptably. In the patent literature, U.S. Pat. No. 5,404,591 to Brinnand exemplifies a well-intentioned but non-optimized glove-style product and mode of treatment. Brinnand includes a pad of material under the heel of the palm to rest and isolate the hand from a desk surface; however the glove does little to restrain wrist flexion and does not adjust in any meaningful fashion to particular positions of angular restraint that will relieve carpal tunnel nerve contact or pressure. Such a product does not address or provide any intervention or remedy for CTS.

Thus it is an objective of the within invention to provide a therapeutic wrist appliance that will allow sufficient hand and finger movement in a working environment.

Another objective of the invention is to restrain wrist movement in a carpal tunnel syndrome sufferer to a position that will relieve pressure on nerve and tendons in the carpal tunnel (neutral position).

Yet another objective is to provide a wrist appliance that is easily put on and removed in order to promote constant or at least frequent use in a working environment.

An additional objective is to provide an appliance that is fully adaptable to a keyboard working environment, including the ability to conveniently adapt to typical support requirements of the hand and forearm relative to a computer keyboard on a flat desktop.

Another objective is to provide an appliance which is fully adaptable to other types of work such as check-out clerks. These and other objectives are met by the invention summarized and described particularly below.

### BRIEF SUMMARY OF THE INVENTION

Restraint of the wrist to the neutral position is critical to treatment and prevention of CTS. In the neutral position, the hand is aligned along the axis of the forearm and pronated or rotated to the palm-down position.

In this posture, the nerves and tendons passing through the carpal tunnel are free of contact with the bony enclosure of the tunnel and thus free from the primary source of abrasion, tension and resulting pain.

One difficulty in achieving the neutral position is that it is inexactly defined and can be different for each sufferer, or even different as between the right and left hand of a single sufferer. Small angular movement or adjustment can be the difference between a fully neutral, relieved posture and a fully contacted, painful posture of the wrist.

Thus each sufferer must find the neutral position by experimentation that best relieves pain, and thus an effective restraining appliance must be flexible enough to accommodate a range of adjustments and yet retain the position found to be most effective by an individual wearer.

The instant invention achieves user restraint to the neutral positions by providing a specially configured carpal tunnel support that includes a foam rubber support panel to stiffen the splint and act as pad under the palm and heel of the wrist.

The support material is a stretch material that is resilient in two directions and can be provided in different degrees of spring strength. The combination of restraining features will allow movement of the fingers, and to a limited degree, the hand and wrist, and gently urge return of the enclosed joint to comfortable neutral position. The deformable nature of the support appliance itself and the slightly deflectable padded batten provides a gentle urging to the correct position rather than a rigid restraint which would destroy working functionality.

The support tube provides the primary degree of adjustability in that the resilient material that comprises the pad, can adjust not only to the shape of the user's angular wrist flexion that defines the maximum point of pain relief, but can also adjust to the environmental surface on which it will generally or occasionally be brought to rest, such as the edge of a desktop surface or the back edge of a computer keyboard.

Also worthy of note is that the support is a substitute for other separate pads that are typically, but also ineffectively in many cases, employed to support the wrist for CTS pain relief such as a rubber pad along the front edge of the keyboard or a separate pad at the pointing device (mouse) station. Thus the invention provides an additional benefit of supporting the user's entire forearm to promote the neutral position as the hand is moved to various operational positions.

### BRIEF DESCRIPTION OF THE DRAWINGS

FIG. 1 is an elevational view, partially in section, of the Carpal tunnel support fitted to a user's arm.

FIG. 2 is an isometric view of the Carpal tunnel support.

FIG. 3 is an end elevational view of the Carpal tunnel support.

FIG. 4 is a plan view of the Carpal tunnel support.

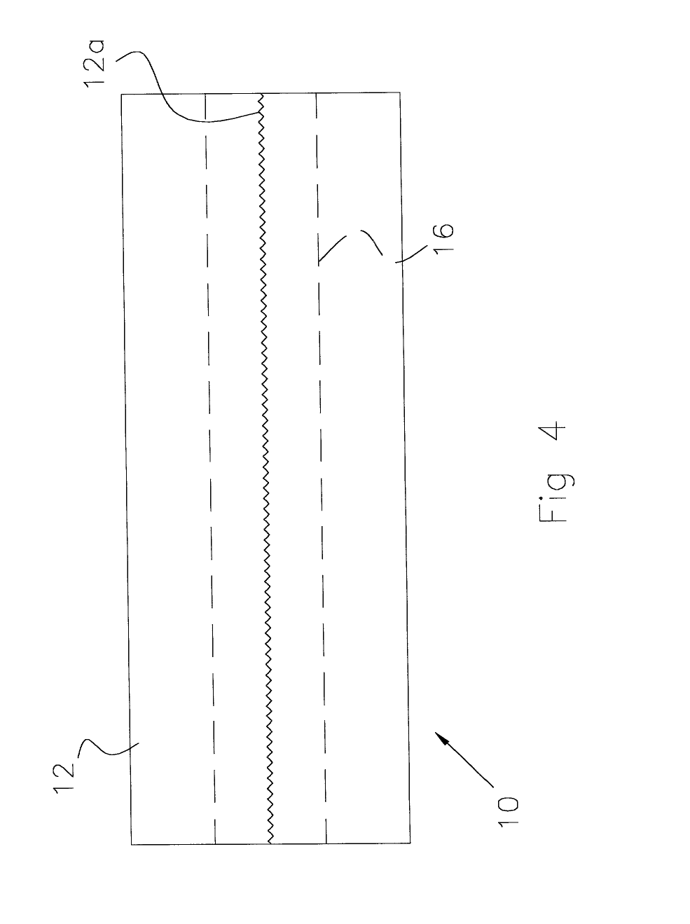

### DETAILED DESCRIPTION OF THE INVENTION

Referring now to the drawings wherein like numerals represent like and corresponding parts throughout the several views in each Figure, the invention is designated overall by the numeral 10.

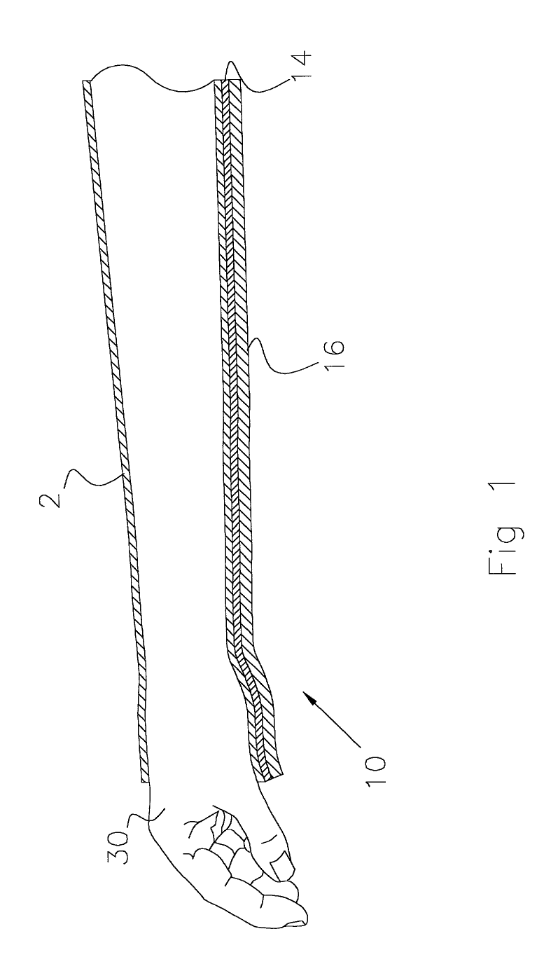

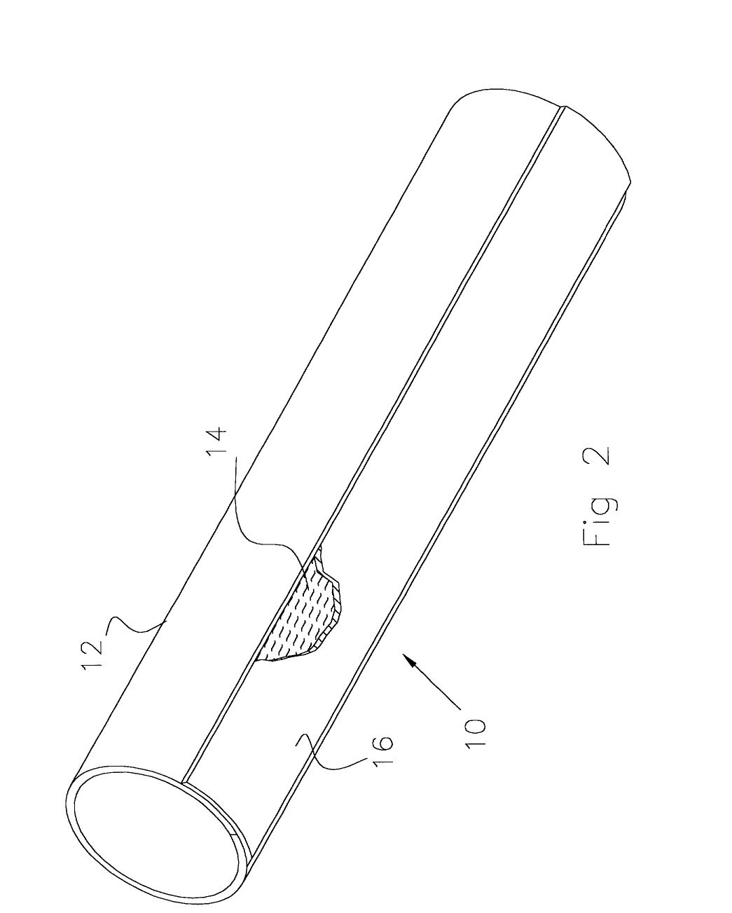

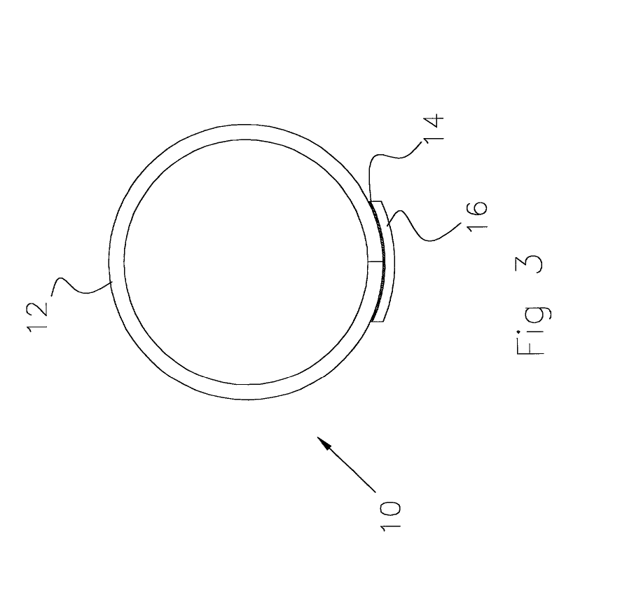

Referring now to FIG. 1, forearm 30 is enclosed within (cover) tunnel 12. Tunnel 12 is selected from a choice of fabrics and preferably nylon, for nylon has durability and stretch ability for wrapping around a wrist of average size. Children's sizes are also included. The support panel 16 is made from a hypo-allergenic foam rubber and in a preferred embodiment, consisted of a panel 2″×2″×12″. Strip 14 consists of hook and loop material (VELCRO) with the hook portion fastened to the support panel 16 and the loop portion fastened to the tunnel 12. FIGS. 2 and 3 show the attachment of support panel 16 to tunnel 12 with strip 14.

In FIG. 5, sewn seam 12a forms tunnel 12. The future position of support panel 16 is shown in dotted lines.

What is claimed is:

1. A Carpal tunnel support for use by a wearer in the operation of components located above a work surface, the support comprising:
a flexible sleeve having a first forward aperture and a second rearward aperture for admitting the hand of a wearer, said sleeve extending to cover the heel of the palm and the wrist portion adjacent to the hand, said first forward aperture for allowing the fingers and the hand to extend outwardly and downwardly in their relaxed state for movement between engagement and disengagement relative to the components, said sleeve having a strip of hook and loop material fastened thereto from said first forward aperture to said second rearward aperture, and
an elongated support panel having a strip of hook and loop material fastened on one side thereof along the entire length of said panel, said support panel hook and loop material being attached to said hook and loop material of said sleeve.

2. A Carpal tunnel support as described in claim 1 wherein said sleeve consists of nylon and said support panel consists of rubber foam material.

3. A carpal tunnel support as described in claim 2 wherein said support panel consists of a 2″×2″×12″ rubber foam material.
